(19) United States
(12) Patent Application Publication
(10) Pub. No.: US 2017/0047709 A1
(43) Pub. Date: Feb. 16, 2017

(54) WAVELENGTH CONTROL OF LASER DIODES

(71) Applicant: Cranfield University, MK43 0AL (GB)

(72) Inventors: Elizabeth Jane Hodgkinson, Cranfield (GB); Ralp Peter Tatam, Cranfield (GB); c/o Abdullah Shah Asmari, Cranfield (GB)

(73) Assignee: Cranfield University, MK43 0AL (GB)

(21) Appl. No.: 15/304,153

(22) PCT Filed: Apr. 9, 2015
(86) PCT No.: PCT/GB2015/051077

§ 371 Date: Oct. 14, 2016

(30) Foreign Application Priority Data
Apr. 14, 2014 (GB) 1406664.1

(51) Int. Cl. H01S 5/068; G01N 21/27; G01N 21/39; H01S 5/024; G01N 33/00

(52) U.S. Cl. CPC H01S 5/06808; H01S 5/02415; H01S 5/06804; G01N 33/0047; G01N 21/39; G01N 21/27; G01N 2021/399; G01N 2201/062

(57) ABSTRACT

This invention generally relates to a method of controlling emission wavelength of a light emitting device, and a system for wavelength control of a light emitting device, for example for wavelength stabilisation of a laser diode. One method of controlling emission wavelength of a light emitting device comprises: determining an indication of series resistance of the device, the series resistance comprising ohmic resistance of the device; providing a current through the device to maintain light emission of the device; measuring a forward voltage of the device during said light emission, the forward voltage being across an impedance comprising impedance of an active region of the device and the series resistance; determining an indicator of active region voltage on the basis of the measured forward voltage and the determined indicator of series resistance; and controlling a temperature of the device on the basis of the determined indicator of active region voltage.

### FIELD OF THE INVENTION

This invention generally relates to a method of controlling emission wavelength of a light emitting device, and a system for wavelength control of a light emitting device, for example for wavelength stabilisation of a laser diode.

### BACKGROUND TO THE INVENTION

Laser diode temperature is a function of ambient temperature, junction heating and thermal design of the laser diode package. Both laser diode threshold current and emission wavelength are functions of temperature. Laser diode temperature stability is of paramount importance in applications such as telecommunications and spectroscopy. High spectral resolution demands the use of singlemode operation and a wavelength-selective element. Conventional lasers used in these applications are typically distributed feedback (DFB) laser diodes, in which the wavelength-selective elements is a grating embedded along or above the active region, or vertical cavity surface emitting lasers (VCSELs), in which the wavelength-selective element is formed from interference layers that lie above and below the active region.

LEDs are often used in lower resolution, non-dispersive spectroscopy. For this technique, two or more broadband spectroscopic channels are defined using narrowband filters, one corresponding to a region of high optical absorption by the target gas (the measurement channel) and the second corresponding to a region of minimal absorption (the reference channel). The reference channel compensates for changes in the emission intensity of the broadband source. Any spectral difference in the source emission, i.e. that affects the measurement and reference channels disproportionately, will be wrongly interpreted as positive or negative optical absorption by the target species and lead to errors in the results. However, it is known that temperature changes to the LEDs can cause a distortion of the spectrum and thereby cause exactly this problem, whether in the UV, visible or infra-red regions of the spectrum. Temperature control of LEDs used in non-dispersive spectroscopy is therefore an important contributor to the limit of detection.

Laser diode operating temperature is controlled conventionally using a thermoelectric cooler (TEC) such as a Peltier element. The temperature of the laser diode is sensed with a thermistor at a short distance from the active region. The temperature precision of this technique is typically 0.01° K. However this technique suffers from reduced accuracy as it senses the temperature of the thermistor not the laser. There is a temperature gradient between the thermistor and the laser, caused by chip heating. Despite the use of good thermal design and a case, a change in ambient temperature may cause a change to the thermal gradient, resulting in a systematic error in the emission wavelength when the ambient temperature (temperature external to the laser package) is varied. A potential alternative technique is to use the voltage drop across the laser diode to measure the temperature of the junction. This technique has been widely used in diode based thermometers for several decades. Thermometers using the junction voltage of a diode can sense temperature in the range of 1K-400K with sensitivity of 1 mK.

The laser diode central wavelength also varies with the injection current. There are two factors at work. (i) Increased heating, following an increase in injection current, causes thermal expansion of the wavelength selective element, resulting in a longer wavelength emission. This junction heating is due to non-radiative processes (where the efficiency of the laser diode in converting electrical energy into light is less than unity) as well as radiative reabsorption of light by the laser diode gain chip. (ii) Changes in injection current cause a change in refractive index of the laser diode cavity. The result is that the laser diode emission wavelength increases with increasing injection current.

Thus, the field of light emitting device control continues to provide a need for improved wavelength control.

For use in understanding the present invention, the following disclosures are referred to:
J. H. Jeong, K. C. Kim, J. L. Lee, H. J. Kim, and I. K. Han. “Junction temperature measurement of InAs quantum-dot laser diodes by utilizing voltage-temperature method”, IEEE Photonics Technology Letters 20 (16), 1354-1356, 2008.
H. Y. Ryu, K. H. Ha, J. H. Chae, O. H. Nam, and Y. J. Park. “Measurement of junction temperature in GaN-based laser diodes using voltage-temperature characteristics”, Applied Physics Letters 87 (9), 093506, 2005.
Y. Xi, T. Gessman, J. Xi, J. K. Kim, J. M. Shah, E. F. Schubert, A. J, Fischer, M. H. Crawford, K. H. A. Bogart and A. A. Allerman. “Junction temperature in ultraviolet light-emitting diodes”, Japanese Journal of Applied Physics 44 (10), 7260-7266, 2005.
J. M. Smith. “Temperature compensation for gas detection” patent: WO/2009/019467
K. Uehara and K. Katakura. “New method of frequency stabilization of semiconductor lasers”, Japanese Journal of Applied Physics 27 (2), 244-246, 1988.

### SUMMARY

According to a first aspect of the present invention, there is provided a method of controlling emission wavelength of a light emitting device, the method comprising: determining an indication of series resistance of the device, the series resistance comprising ohmic resistance of the device; providing a current through the device to maintain light emission of the device; measuring a forward voltage of the device during said light emission, the forward voltage being across an impedance comprising impedance of an active region of the device and the series resistance; determining an indicator of active region voltage of the device on the basis of the measured forward voltage and the determined indicator of series resistance; and controlling a temperature of the device on the basis of the determined indicator of active region voltage.

Thus, in an embodiment, there is provided a method of controlling emission wavelength of a light emitting device, the method comprising: determining an indication of series resistance of the device; providing a current through the device to maintain light emission of the device; measuring a forward voltage of the device during said light emission, the forward voltage being across an impedance comprising the active region and associated contacts; determining an indicator of the active region voltage on the basis of the measured forward voltage and the determined indicator of series resistance; and controlling a temperature of the device on the basis of the determined indicator of active region voltage.

For an embodiment, an active region voltage may be termed a voltage across an active region of the device.

The active region voltage may comprise the voltage resulting from the active region, and for example might be termed the junction voltage for an active device comprising a pn junction. The series resistance may comprise the effective ohmic resistance of the device in an equivalent circuit, comprising not only the resistance of ohmic contacts but also the ohmic resistance of the active region of the device, such that the device behaves as though it has a small resistance in series with the active region, although elements of the series resistance may be co-located with the active region. (Since such behaviour may occur in an embodiment, the subject resistance is preferably termed ‘series’ resistance). The forward voltage may comprise the voltage that is measured across the device, which comprises the active region voltage as well as the series resistance multiplied by the injection current.

The series resistance may comprise inherent (intrinsic) resistance of the device and/or comprise resistance of ohmic contacts of the device.

Advantageously, wavelength (e.g., central, average or peak wavelength) of light emission from the device such as a laser diode may thus be accurately set and/or stabilised (when preferably driven at substantially, e.g., exactly, constant current, which may be referred to as an injection current in embodiments), even where an internal and/or external temperature of the light emitting device varies. In an embodiment, the active region voltage indication effectively allows the temperature of the active region (e.g., p-n semiconductor junction (preferably, a depletion region thereof), or quantum dot) of the device, rather than the temperature of the device as a whole, to be controlled to thereby set and/or stabilise the emission wavelength. Such temperature control may compensate for the effect on wavelength of heating of the active region of the device by injection current and/or for the effect on wavelength of ambient temperature changes. In embodiments, the light emitting device may be a quantum cascade laser (such a laser may not strictly be described as a diode) or an interband cascade laser, or broadband (LED-like) versions thereof.

There may further be provided the method, wherein the determining the indication of series resistance comprises: providing an amplitude modulated said current through the device to maintain light emission; extracting an AC component of forward voltage of the device while providing the amplitude modulated current; determining a dynamic resistance indication on the basis of the extracted AC component and the modulation amplitude of the amplitude modulated current, to thereby obtain the indication of series resistance.

For example, the determining the indication of series resistance comprises: providing an amplitude modulated current through the device to maintain light emission at a frequency f; extracting an AC (abbreviated from the well-known term ‘alternating current’) component (or component having a frequency that may be referred to as a high frequency; the AC and/or high frequency component preferably a first harmonic of the modulation frequency f) of forward voltage of the device while providing the amplitude modulated current; detecting a DC (abbreviated from the well-known term ‘direct current’) component (or component at a frequency less than f, preferably much less than f) of forward voltage of the device while providing the amplitude modulated current; determining the series resistance indication on the basis of the extracted AC component and the known amplitude of the applied AC current. Such amplitude modulation may be sinusoidal or may have any other periodic waveform, e.g., pulsed, ramping etc. The extracted AC component generally corresponds to, e.g., is at the same frequency as, the amplitude modulation of the current. Preferably, the modulation depth is less than 5%, preferably less than 3%, more preferably less than 1% or 0.5% of the DC amplitude of the current.

Such an embodiment may comprise inputting the forward voltage to a phase-sensitive detector to perform said extracting the AC component of forward voltage. Such a detector may be a lock-in amplifier.

There may further be provided the method, wherein the indication of series resistance is a voltage (for example, the product of current through the device and the series resistance) and: the measuring the forward voltage of the device comprises detecting a DC forward voltage of the device; the indicator of active region voltage is determined on the basis of the difference between the detected DC forward voltage and the voltage indication of series resistance. (The voltage indication of series resistance may need to be scaled). The detected DC forward voltage may be the maximum size of direct current signal detectable within the forward voltage. The detected DC forward voltage may be obtained by filtering out any AC component from the forward voltage.

There may further be provided the method, comprising: comparing the measured forward voltage and the indication of the series resistance; comparing a result of the comparison to a predetermined value, to thereby generate an error signal; performing said controlling the temperature on the basis of the error signal. The comparing the measured forward voltage and the indication of the series resistance may obtain the difference between the forward voltage and the voltage product of the current and series resistance. The predetermined value is preferably an established forward voltage setpoint or threshold.

There may further be provided the method, wherein a maximum wavelength drift of the device, when the ambient temperature of the device varies between 15 and 35 degrees Celsius, is less than +−0.03 pm/degree Celsius, preferably less than +−0.02 pm/degree Celsius. (This is achievable for example using an InP ridged waveguide laser and operating wavelength of 1650 nm). The wavelength drift may be considered as an ambient temperature coefficient of standard deviation of emission wavelength (preferably central and/or peak) of the laser diode. Any said standard deviation is preferably measured over a fixed period, e.g., 1 hour, during which the DC component of the current through the device is substantially constant.

There may further be provided the method, wherein a standard deviation of emission wavelength (preferably central and/or peak) of the device is less than +−0.7 pm, preferably less than +−0.5 pm or +−0.3 pm, at an ambient temperature of 20 degrees Celsius. Again, any said standard deviation is preferably measured over a fixed period, e.g., 1 hour, during which the DC component of the current through the device is substantially constant.

There may further be provided the method, wherein a maximum variation of standard deviation of emission (preferably central and/or peak) wavelength of the device is less than +−0.6 pm, preferably less than +−0.4 pm, when ambient temperature of the device is varied between 15 and 35 degrees Celsius.

There may further be provided the method, wherein the light emitting device comprises a light emitting diode or laser, preferably a distributed feedback laser and/or comprising a distributed Bragg reflector. For example, the device may be an UV LED (Ultraviolet Light Emitting Diode), an InP and/or GaN laser, and/or a VCSEL (Vertical Cavity Surface Emitting Laser).

Three may be further provided the method, wherein compensation for the effect of series resistance is provided by a reading from a thermistor, such a reading displaying a drift of wavelength with respect to temperature that opposes the drift of wavelength with respect to temperature of the measured forward voltage.

According to another aspect of the invention, there is provided a system for wavelength control of a light emitting device, the system comprising: a current driver to provide an amplitude modulated current through the device to maintain light emission of the device; a thermoelectric element for controllable heating and cooling of the device; a DC voltage detector to detect a DC component of forward voltage of the device; a phase-sensitive detector to extract an AC component of the forward voltage; a comparator to compare the detected DC component to the extracted AC component, to thereby indicate an active region voltage of the device; and a temperature controller configured to control the thermoelectric element on the basis of the indication of active region voltage.

The thermoelectric element may for example be a Peltier element.

The current provided through the device is preferably to bias the device with a DC injection current, while further comprising an AC component to allow the phase-sensitive detection to occur.

There may further be provided the system, wherein the temperature controller comprises a proportional integral and derivative controller configured to control the thermoelectric element on the basis of the indication of active region voltage.

There may further be provided the system, wherein the light emitting device comprises a light emitting diode or laser, preferably a distributed feedback laser and/or comprising a distributed Bragg reflector.

There may be provided a spectrometer comprising the system, the spectrometer preferably for detecting one or more gases having absorption spectra that fall within a range of wavelengths emitted by the device, for example for detecting methane.

According to a further aspect of the invention, there is provided a method of controlling emission wavelength of a light emitting device, the method comprising: providing a current through the device to maintain light emission of the device; measuring a forward voltage of the device during said light emission, the forward voltage being across an impedance comprising an active region of the device and series resistance of the device; determining an indication of temperature of a temperature sensitive device thermally coupled to the light emitting device; determining a corrected forward voltage on the basis of the measured forward voltage and the determined indicator of temperature; and controlling a temperature of the light emitting device on the basis of the determined corrected forward voltage.

The thermal coupling may be achieved by positioning the temperature sensitive device close to the light emitting device.

In an embodiment of the aspect, for the effect of series resistance is provided by a reading from a thermistor, such a reading displaying a drift of wavelength with respect to temperature that opposes the drift of wavelength with respect to temperature of the measured forward voltage.

Since the thermistor reading and the forward voltage reading in an embodiment may have opposite effects on the wavelength with respect to changes in external temperature, therefore if the controller is based on adding the readings from both in appropriate proportions, this may compensate both measurements appropriately and give a stable wavelength.

According to another aspect of the invention, there is provided a system for wavelength control of a packaged light emitting device, the system comprising: a temperature sensitive device thermally coupled to the device package; a DC voltage detector to detect a DC component of forward voltage of the device; a comparator to compare a reading from the temperature sensitive device to the detected DC component to give a corrected DC forward voltage; and a temperature controller configured to control the thermoelectric element on the basis of the indication of corrected DC forward voltage, to thereby control temperature of the light emitting device.

The temperature sensitive device may comprise a thermistor, and/or may be embedded within the device package or positioned close to the outside of the package.

In an embodiment of the aspect, for the effect of series resistance is provided by a reading from a thermistor, such a reading displaying a drift of wavelength with respect to temperature that opposes the drift of wavelength with respect to temperature of the measured forward voltage.

Preferred embodiments are defined in the appended dependent claims.

Any one or more of the above aspects and/or any one or more of the above optional features of the preferred embodiments may be combined, in any permutation.

### BRIEF DESCRIPTION OF THE DRAWINGS

For a better understanding of the invention and to show how the same may be carried into effect, reference will now be made, by way of example, to the accompanying drawings, in which:

FIG. 1 shows laser diode centre wavelength stability with ambient temperature under junction voltage control;

FIG. 2 shows a 3D model of a DFB laser diode in a butterfly package;

FIG. 3 shows an investigation of conventional method of laser diode wavelength stabilisation;

FIG. 4 shows laser diode wavelength drift with ambient temperature while conventionally controlled to a thermistor temperature of 15° C., at constant injection current of 150 mA;

FIG. 5 shows a “See-saw” model of conventional laser diode control, using a thermistor set point;

FIG. 6 shows laser diode long term wavelength stability at constant ambient temperature of 30° C., thermistor temperature of 15° C. and constant injection current of 150 mA;

FIG. 7 shows a flow chart showing forward voltage based control of Peltier current;

FIG. 8 shows a schematic of forward voltage method to stabilise laser diode wavelength;

FIG. 9 shows laser diode wavelength stability with variable ambient temperature under forward voltage control;

FIG. 10 shows a laser diode long term wavelength stability test using forward voltage based control;

FIG. 11 shows an example laser diode small signal model;

FIG. 12 shows laser diode series resistance measurement operating under thermistor based control at 10° C.;

FIG. 13 shows a V-I plot of the laser diode operating under thermistor based control at different temperatures;

FIG. 14 shows a flow chart for laser diode wavelength stabilisation using junction voltage;

FIG. 15 shows configuration of junction voltage method for stabilising the wavelength of the laser diode;

FIG. 16 shows a methane 3f signal from a gas cell used for wavelength measurement;

FIG. 17 shows an expanded section of FIG. 16, showing a linear region around the line centre. The equation of a best fit straight line through the experimental points is also given;

FIG. 18 shows long term wavelength stability for the laser diode using junction voltage based control;

FIG. 19 shows laser diode wavelength deviation from the methane line centre (1650.96 nm) under junction voltage control, at ambient temperatures of 15° C. and 35° C.;

FIG. 20(a-c) show Labview code used for laser diode wavelength stability using forward voltage based control.

### DETAILED DESCRIPTION OF PREFERRED EMBODIMENTS

Generally, embodiments provide wavelength control of laser diodes via measurement of junction voltage, also termed active region voltage of the laser diode. Specifically, wavelength stabilisation may be achieved in an embodiment by using the voltage drop across a laser diode to measure the temperature of the active medium instead of a thermistor. Advantageously, the junction voltage rather than the forward voltage is used. Since the series resistance of these devices imposes a small but significant error when measuring the forward voltage, by measuring this resistance and compensating for it, the junction voltage alone is measured, the accuracy of the measurement is improved significantly and/or the emission wavelength is stabilised, preferably with no measurable systematic error when the ambient temperature is varied in the range 15-35° C. The junction temperature may be measured more accurately, and/or the effects of ambient temperature may be compensated.

To aid understanding, we now consider firstly the V-I relationship of a laser diode.

The injection current and laser diode junction voltage can be represented by Shockly equation

$${I}_{f} = {I}_{s} exp ( \frac{{eV}_{j}}{η kT} ) \qquad ( 1 )$$

Where I_{f} and Iₛ are forward and saturation current of the laser diode respectively. Vⱼ is the junction voltage of the laser diode, e is the electronic charge, k is the Boltzmann's constant, and T is the thermodynamic temperature in degrees Kelvin. η is the ideality factor and is approximately equal to 1 for a laser diode operated above the threshold.

The voltage of the laser diode can be derived from the Shockly equation

$${V}_{j} = ln \frac{{I}_{f}}{{I}_{s}} \frac{η kT}{e} \qquad ( 2 )$$

### When

$$\frac{η kT}{e} ≪ {V}_{j} ,$$

which is also called the thermal voltage then

$$\begin{matrix}\frac{d {V}_{j}}{{d}_{T}} = \frac{d}{d T} [ \frac{η kT}{e} ln \frac{{I}_{f}}{{I}_{s}} ] \\ Where & ( 3 ) \\ {I}_{s} = [ {T}^{3} exp ( \frac{- {E}_{g}}{kT} ) ] {T}^{\frac{γ}{2}} & ( 4 )\end{matrix}$$

E_{g} is the band gap energy measured in volts and γ is a constant. The

$${T}^{\frac{γ}{2}}$$

term is close to unity, and the saturation current can be approximated as

$${I}_{s} = {T}^{3} exp ( \frac{- {E}_{g}}{kT} ) C \qquad ( 5 )$$

where C is a constant. Substituting equation (5) in equation (3),

$$\frac{d {V}_{j}}{{d}_{T}} = \frac{d}{d T} [ \frac{η kT}{e} ln ( \frac{{l}_{f}}{C} ) - \frac{3 ln T η kT}{e} + \frac{η {E}_{g}}{e} ] \qquad ( 6 )$$

The above relationship gives the temperature dependence of the laser diode junction voltage. The forward voltage V_{f} of the laser can be described as

V_{f}=Vⱼ+I_{f}Rₛ (7)

V_{f} and Rₛ are the voltage measured across the terminals of the laser diode and the series resistance of the laser diode. The derivative of the junction voltage in terms of forward voltage can be written as

$$\frac{d {V}_{j}}{{d}_{T}} = \frac{d}{d T} [ \frac{η kT}{e} ln ( \frac{{l}_{f}}{C} ) - \frac{3 ln T η kT}{e} + \frac{η {E}_{g}}{e} ] - \frac{d}{d T} ( {I}_{f} {R}_{s} ) \qquad ( 8 )$$

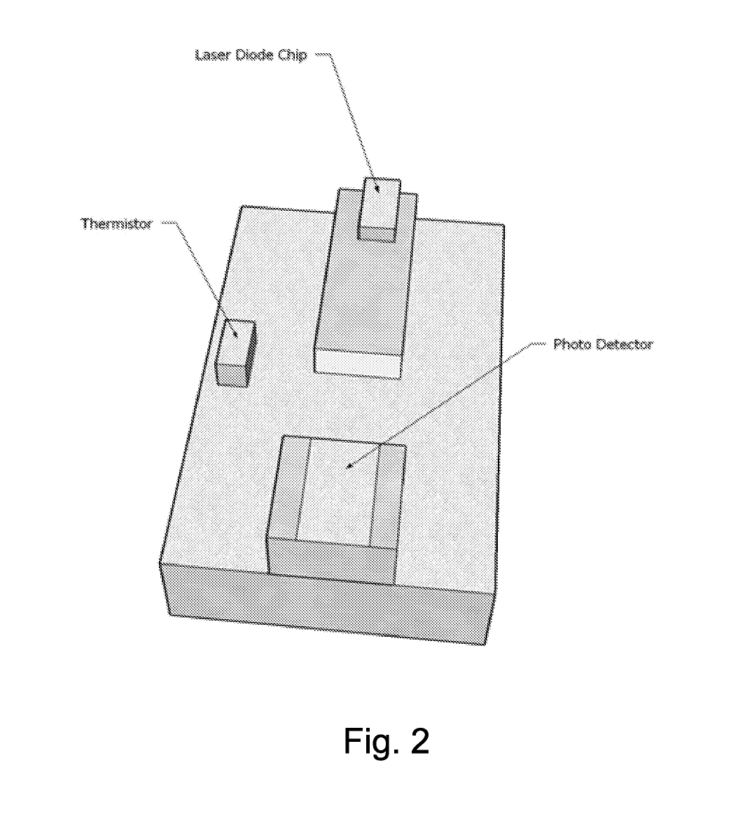

Considering now laser diode wavelength stability using conventional thermistor based control, it is noted that, in a conventional laser diode temperature control system, the temperature of the laser diode chip is sensed with a thermistor placed at a distance as shown in FIG. 2. The thermistor gives a temperature measurement of its immediate surroundings.

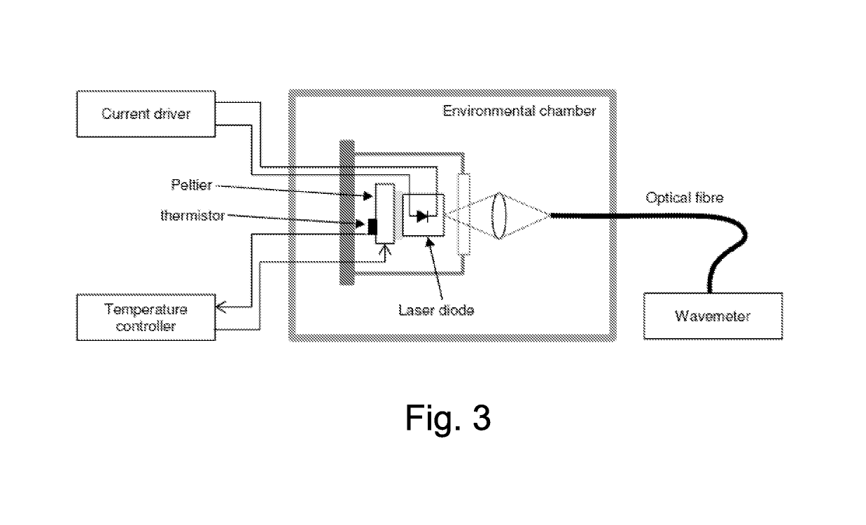

Regarding equipment and experimental configuration, an InP ridged waveguide laser with a wavelength of 1650 nm (HHI TO8 MTE Module) was investigated for wavelength stability using conventional thermistor-based control. The thermistor inside the laser diode package was connected to a temperature controller (Thorlabs TED200), which in turn was connected to the Peltier element in the package in order to control the thermistor temperature. The injection current to the laser diode was supplied using a current driver (Thorlabs LDC200). An environmental chamber was used to maintain constant external ambient temperature to within ±1° C., as shown in FIG. 3. The wavelength of the laser diode was measured with a wavelength meter (Agilent AG86122B). The resolution of this wavemeter was 100 fm and its absolute accuracy was 0.3 pm. Because the wavemeter could not measure wavelengths longer than 1650 nm, for this test the laser diode was operated at 1649 nm.

The laser diode was operated above threshold at different ambient temperatures, placed in an environmental chamber in the range of 15−35° C. The laser diode injection current was kept constant at 150 mA±0.5 mA and the thermistor temperature was maintained at 15° C.±0.01° C.

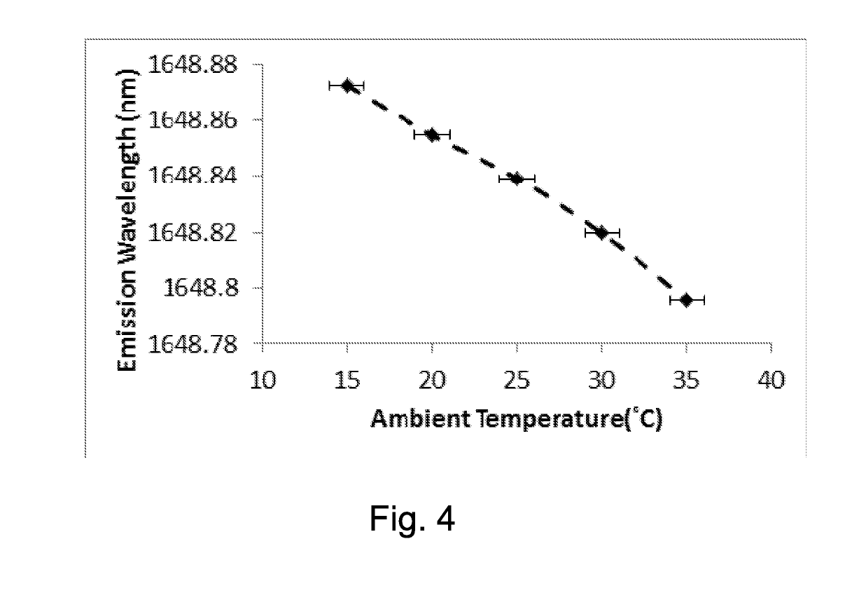

Considering the results for conventional control, FIG. 4 demonstrates the effect of variable ambient temperature on the laser diode wavelength. The ambient temperature for the laser diode was varied in 5° C. steps by changing the environmental chamber temperature.

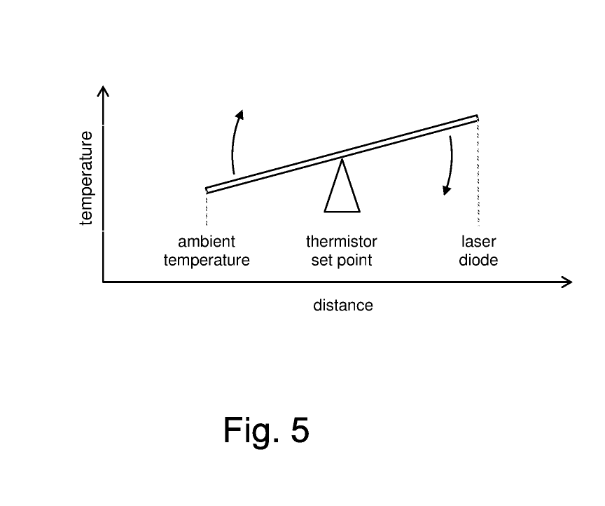

As the ambient temperature was increased from 15° C. to 35° C., the laser diode emission wavelength drifted to shorter wavelengths, despite having constant injection current and thermistor temperature. This is consistent with the hypothesis that the thermistor was not measuring the actual temperature of the laser diode and as the ambient temperature was increased, the temperature gradient between the chip and the thermistor was affected. Therefore, the laser diode chip was further cooled down. FIG. 5 illustrates this principle with a simple “see-saw” model.

Thus, the laser diode wavelength drifted towards shorter wavelengths with a total wavelength shift of 77 pm over the temperature range of 15-35° C. The change in laser diode wavelength with ambient temperature was −3.8 pm±0.5 pm/° C. at a constant thermistor temperature.

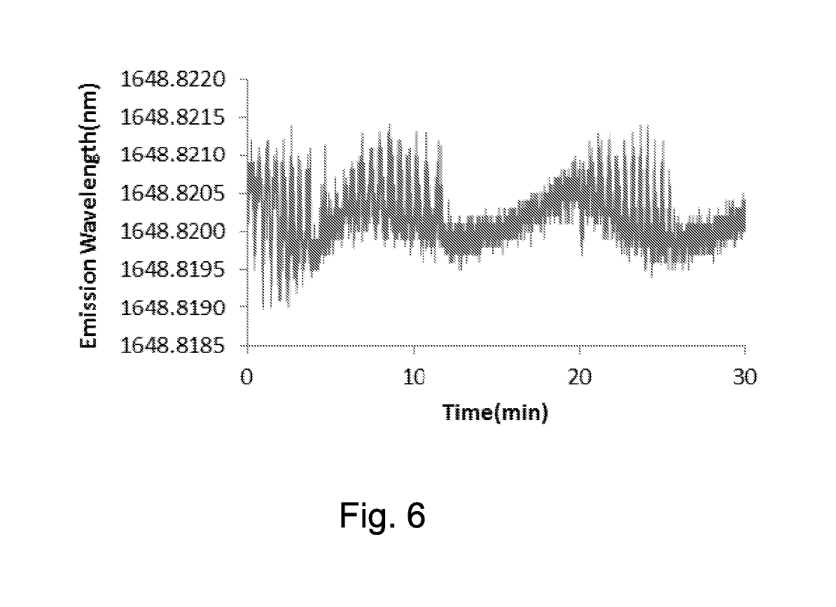

The conventional thermistor control method was also tested for long term laser wavelength stability. The injection current to the laser diode was kept constant at 150 mA±0.5 mA and the thermistor temperature was controlled to 15° C.±0.01° C. The ambient temperature was set to 30° C. and the wavelength monitored for 30 min. FIG. 6 shows the resulting long term wavelength stability. The laser diode wavelength remained stable over time with a standard deviation of σ=±0.4 pm over a time of 30 minutes.

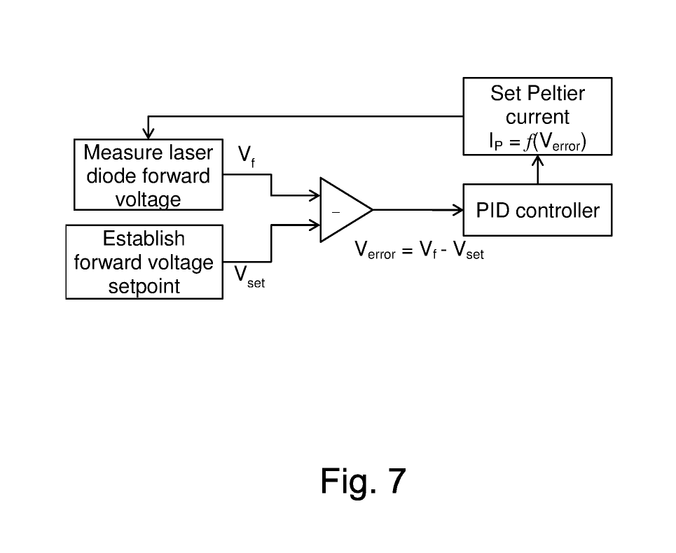

Turning now to an assessment of laser diode wavelength stability using forward voltage based control, the laser forward voltage was used as a temperature sensor to stabilise the wavelength of the laser diode. The equipment and experimental setup involved the same DFB laser used in the above described investigation of conventional method of laser diode wavelength stabilisation. The laser was investigated for wavelength stability using a forward voltage method. The laser diode was driven using the same current driver (ThorLabs LDC200) and its emission was monitored using the same wave meter (Agilent AG86122B) as described in the above discussion of the investigation of conventional method of laser diode wavelength stabilisation. The laser diode was again placed in an environmental chamber whose temperature could be changed in the range 15-35° C. A Labview (National Instruments) based proportional integral and derivative (PID) controller was used to implement a forward voltage based temperature controller, as shown in the flowchart in FIG. 7.

The laser diode was driven with a constant injection current of 150 mA±0.5 mA. The voltage drop measured while the thermistor was controlled to 15° C. was used as setpoint voltage for the PID controller. The ambient external temperature of the environmental chamber was varied in steps of 5° C. over the range 15-35° C.

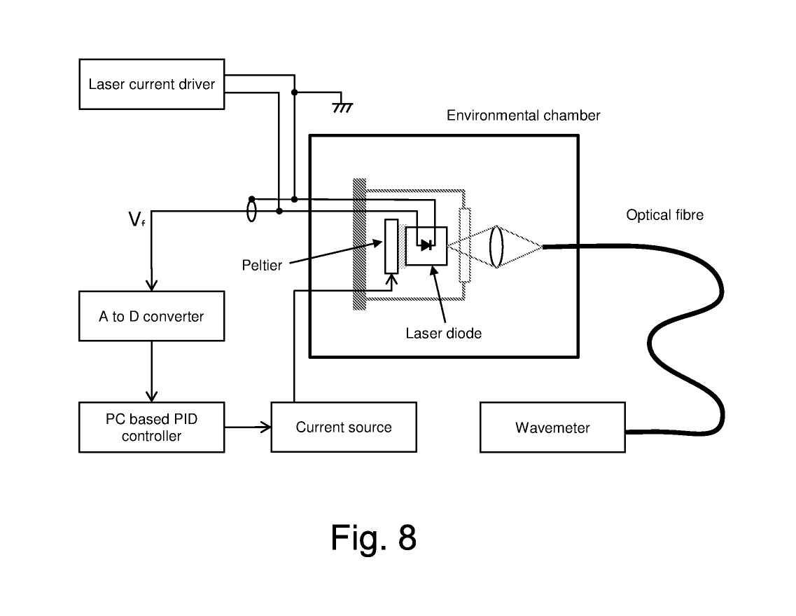

The voltage drop across the laser diode was measured and compared to the voltage setpoint. The difference between these voltages acted as an error signal, which was fed into the PID controller to stabilise the temperature of the laser diode as described in FIG. 8.

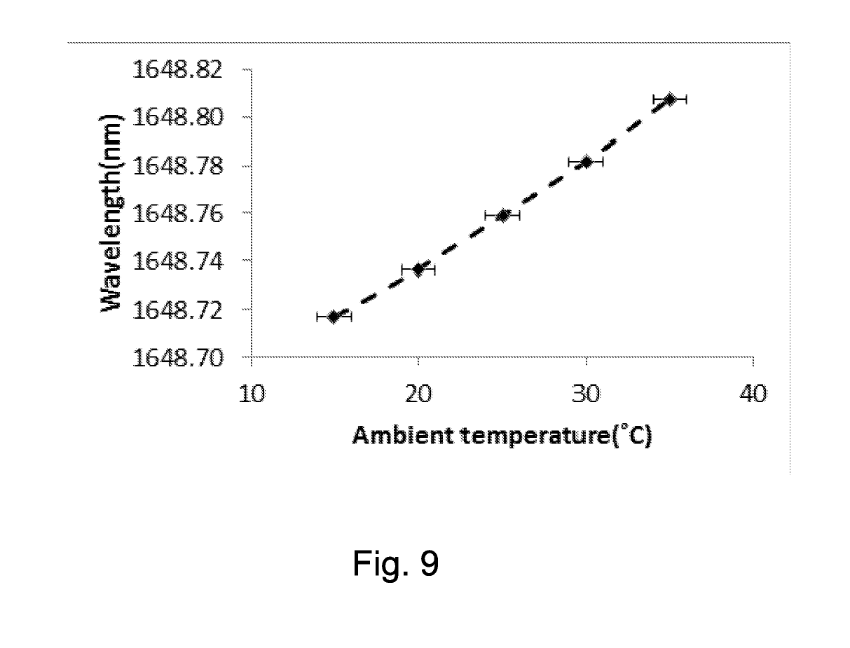

Regarding the results, FIG. 9 shows the relationship between ambient temperature and laser diode emission wavelength under forward voltage control. The laser diode wavelength was found to drift towards longer wavelengths at higher ambient temperatures, in contrast to the drift observed using the conventional method, where the wavelength became shorter as described in section (3.2). It was found that this technique suffered from a systematic error of 4.5 pm/° C. with changes in ambient temperature, as can be observed in the graph in FIG. 9.

FIG. 9 shows that the laser diode wavelength drifted towards longer wavelengths at higher ambient temperatures, indicating that the forward voltage is not the only important parameter for measurement of laser junction temperature. The total wavelength drift was 90 pm over the range 15-35° C. The performance of the forward voltage method in stabilising the wavelength of the laser diode with variable ambient temperature is therefore comparable to that of conventional thermistor based control.

The forward voltage technique was also tested for long-term wavelength stability. The laser diode was kept in an environmental chamber at a constant temperature of 30° C.±1° C. The laser diode chip was biased with an injection current of 150 mA±0.5 mA and the wavelength was measured with the wavemeter (described above in relation to the conventional method of laser diode wavelength stabilisation) over a period of 30 minutes.

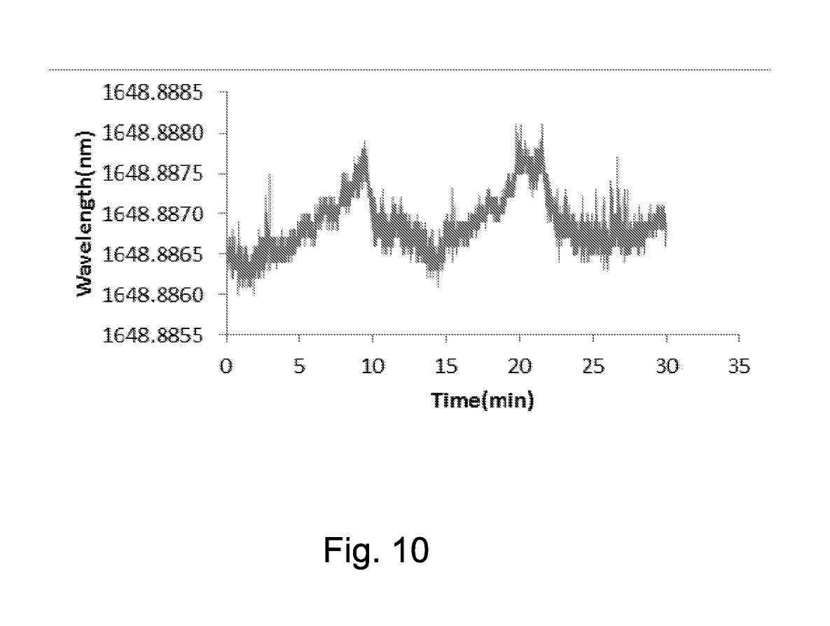

FIG. 10 shows the long term wavelength stability of the laser diode at constant temperature and injection current. The wavelength stability was ±0.32 pm (1σ), which again is comparable to conventional thermistor based control.

The long-term wavelength stability and systematic error with ambient temperature changes were comparable for temperature control using the forward voltage method and using the thermistor. However, tunable diode laser spectroscopy of gases such as methane, where absorption line the half width half maximum is 50 pm (100% methane concentration at atmospheric pressure), requires a more stable laser diode wavelength when faced with varying ambient temperatures. Therefore, an improved wavelength control method is desirable.

We now consider laser diode junction voltage based control. The forward voltage is the voltage measured across the anode and cathode of the laser diode. The forward voltage is the sum of junction voltage and the voltage drop across the series resistance of the laser diode, Rₛ as described in equation (7). Both junction voltage and series resistance are temperature dependent as shown in equation (8). As described in the following, the effect of temperature on forward voltage and series resistance are investigated. The laser diode junction voltage is investigated for the purpose of stabilising the wavelength of the laser diode over long timescales and in the face of variable ambient temperature.

The laser diode series resistance can be measured as follows. From the first derivative of the Shockley equation (1) combined with equation (7), and assuming V_{f}>>V_{T}, we have

$$\frac{d {V}_{f}}{d {I}_{f}} = ( \frac{kT η}{q} ) \cdot \frac{1}{{I}_{f}} + {R}_{s} \qquad ( 9 )$$

Which can be rewritten as

R=Rⱼ+Rₛ (10)

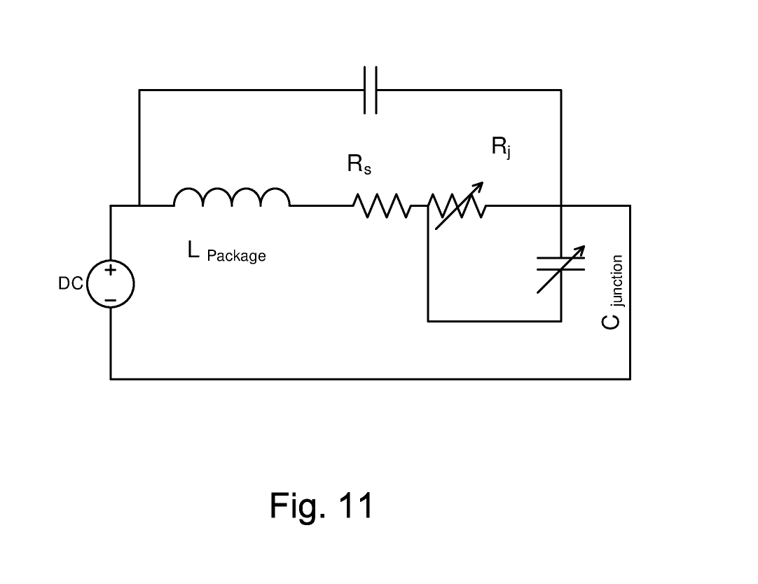

R and Rⱼ are the total dynamic resistance and effective junction resistance of the laser diode respectively. Rₛ is the additional series resistance of the laser diode caused by ohmic effects within the device. FIG. 11 shows the small signal model of a laser diode, which may be an equivalent circuit that may be used to understand how the laser diode behaves.

The laser diode series resistance Rₛ increases with increasing temperature. To determine the value of Rₛ, the forward voltage of the laser diode was measured for a range of injections currents at different operating temperatures. By plotting

$$\frac{d {V}_{f}}{d I}$$

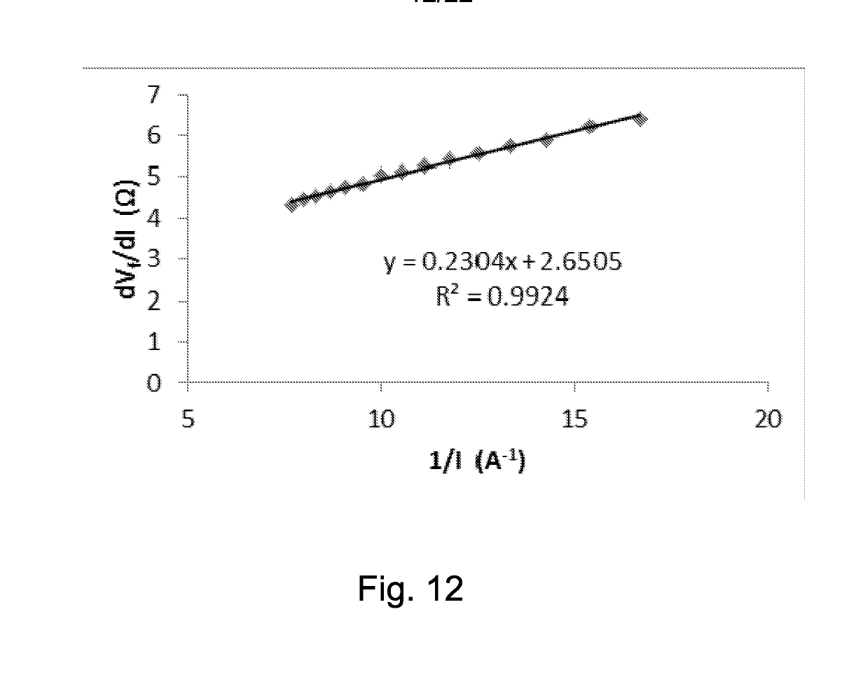

against the injection current (I), the laser diode resistance can be measured as shown in FIG. 12.

FIG. 12 shows the laser diode series resistance at 10° C.; the plot shows a linear relationship between

$$\frac{d {V}_{f}}{d I}$$

and the inverse of the injection current. The y-axis intercept of this graph gives the measurement of laser diode series resistance, which increases with increasing thermistor temperature as given in Table 1. We have found that the temperature dependence of Rₛ in equation (10) is significant, and requires compensation if the forward voltage is to be used for temperature control purposes. Measurement of the dynamic resistance as

$$\frac{d {V}_{f}}{d I}$$

in equation (u) gives a value whose variation with temperature is substantially influenced by that of the series resistance Rₛ, and this can therefore can be used to provide this compensation.

TABLE 1
The effect of temperature on the laser diode series resistance Rₛ

| Operating temperature (° C.) | Laser Diode Resistance Rₛ (Ω) |
|---|---|
| 10 | 2.65 |
| 20 | 2.70 |
| 30 | 2.74 |
| 40 | 2.86 |

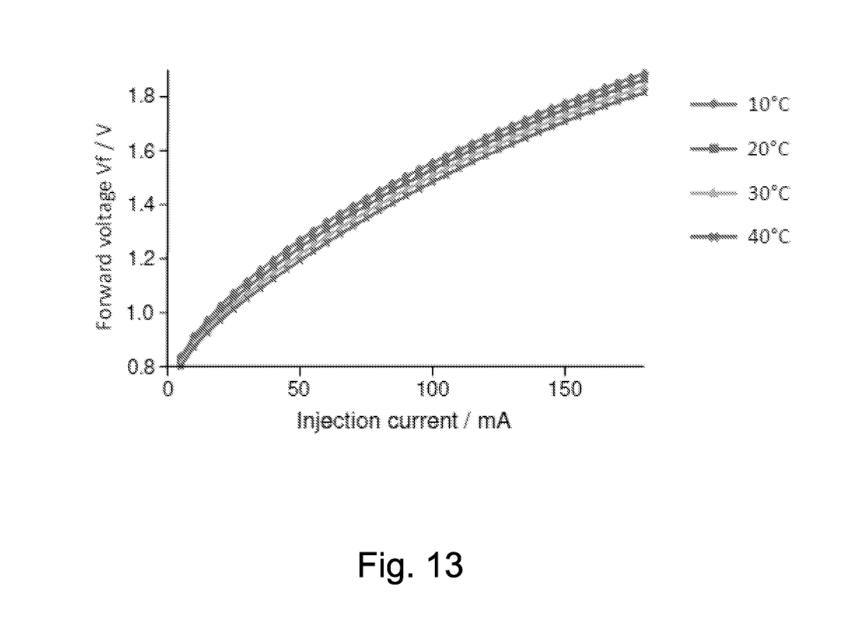

A plot of voltage versus injection current over the temperature range 10-40° C. in FIG. 13 shows that with increasing temperature, the forward voltage decreases, in contrast to the series resistance of the laser diode as shown in Table 1 which increases.

The graphs in FIGS. 12 and 13 confirm that there is a small additional series resistance within the laser diode, which is temperature dependent as shown in Table 1. The voltage drop due to the laser diode series resistance will contribute to the forward voltage by adding a small voltage that is dependent on both temperature and injection current. Therefore, this potentially adds a systematic error to the measurement of temperature of the laser diode using the forward voltage of the laser diode. This systematic error in temperature sensing using forward voltage can be observed in FIG. 9, where the laser diode emission has drifted towards longer wavelengths due to the increasing ambient temperature.

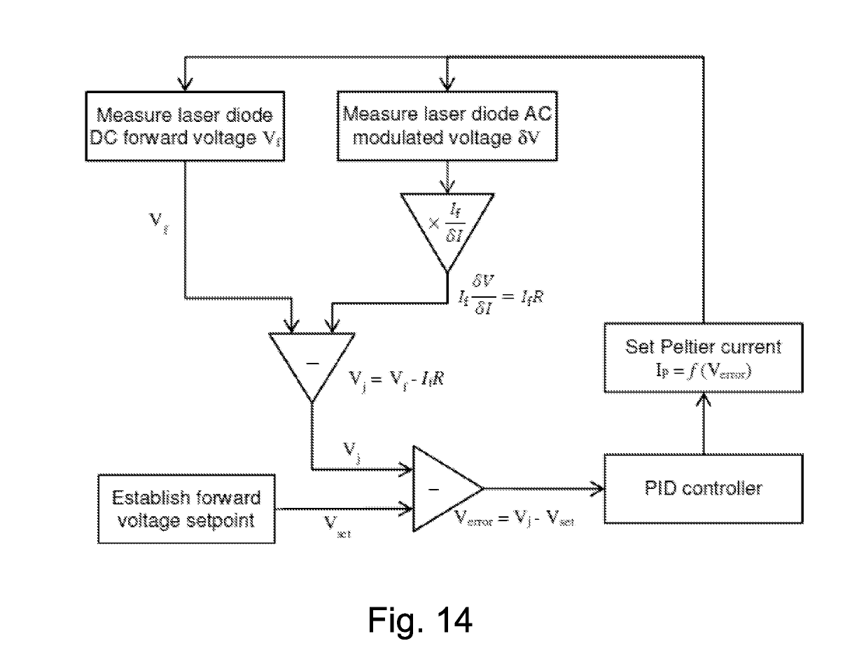

Regarding equipment and experimental setup, the flow chart in FIG. 14 shows a process of stabilising the laser diode wavelength using voltage across the active region of the device (e.g., laser diode junction voltage, the junction generally being a semiconductor pn junction) as opposed to merely forward voltage (forward voltage generally being that dropped between the anode and cathode of the device, e.g., across the impedance represented by the full equivalent circuit of FIG. 11). The laser diode dynamic resistance R was measured by modulating the laser diode using a relatively small modulation depth sinusoidal current (which may be termed an amplitude modulated current), while simultaneously applying a DC injection current above the threshold. The voltage drop across the laser diode was fed into a lock in amplifier and demodulated at 1f. The demodulated signal gave a measure of the laser dynamic resistance R as ∂V_{f}/∂I_{f} at the DC current. In an embodiment, a phase-sensitive detector in the form of a lock-in amplifier is used to extract the AC component δV from the detected forward voltage V_{f}, the extracted signal being processed (e.g., scaled by dividing by the modulation amplitude δI of the modulated current and/or multiplying by the DC amplitude I_{f} of the modulated current), preferably to provide the measure of the laser resistance as ∂V_{f}/∂I_{f}.

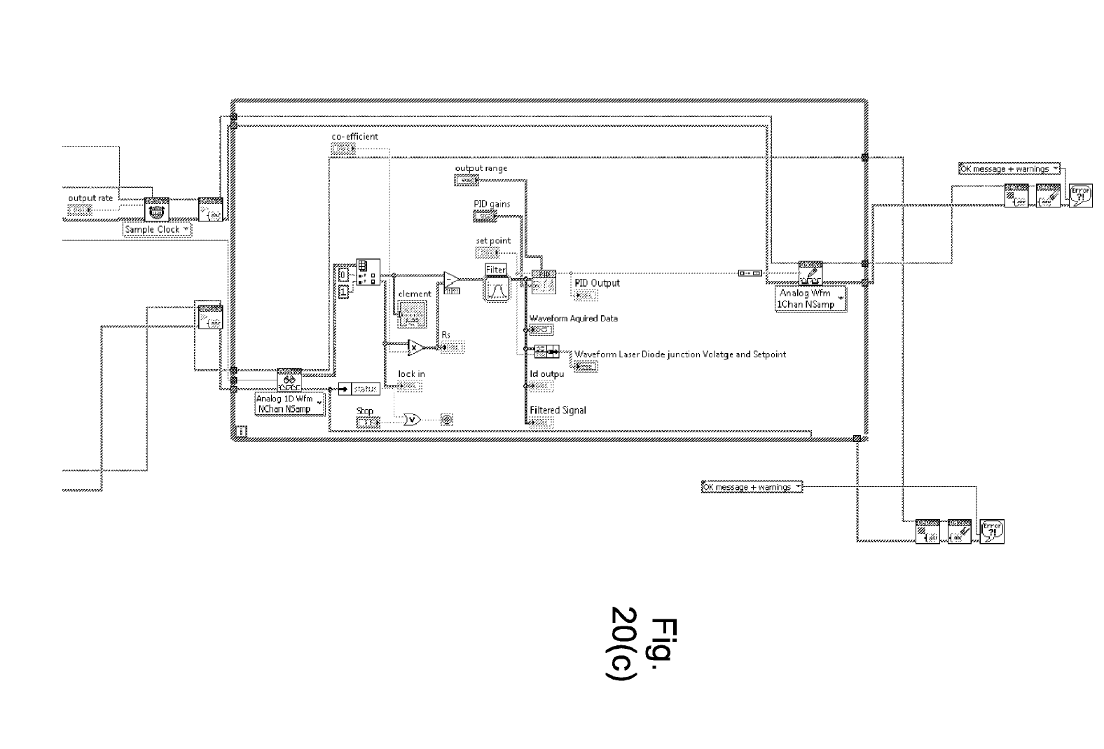

The same PID controller was used as described above in relation to the equipment and experimental setup for laser diode wavelength stability using forward voltage based control. The Labview code (FIG. 20) used for the above experimental setup for laser diode wavelength stability using forward voltage based control was modified as follows. The RMS modulated voltage was measured using a lock-in amplifier and multiplied by an empirically determined factor (I/δI in FIG. 14) to give an indication of the additional voltage due to the laser resistance (IR). This was subtracted from the DC forward voltage to give an indicator of the active region voltage in the form of a measure of the junction voltage Vⱼ. A setpoint value Vₛₑₜ was subtracted from Vⱼ to provide an error signal in the PID feedback loop to the Peltier element.

In practice, the scale factors shown in FIG. 14 are used to give a suitable starting point for the voltage compensation, and are then adjusted empirically to take account of the minor effects of Rⱼ and any additional ohmic contacts.

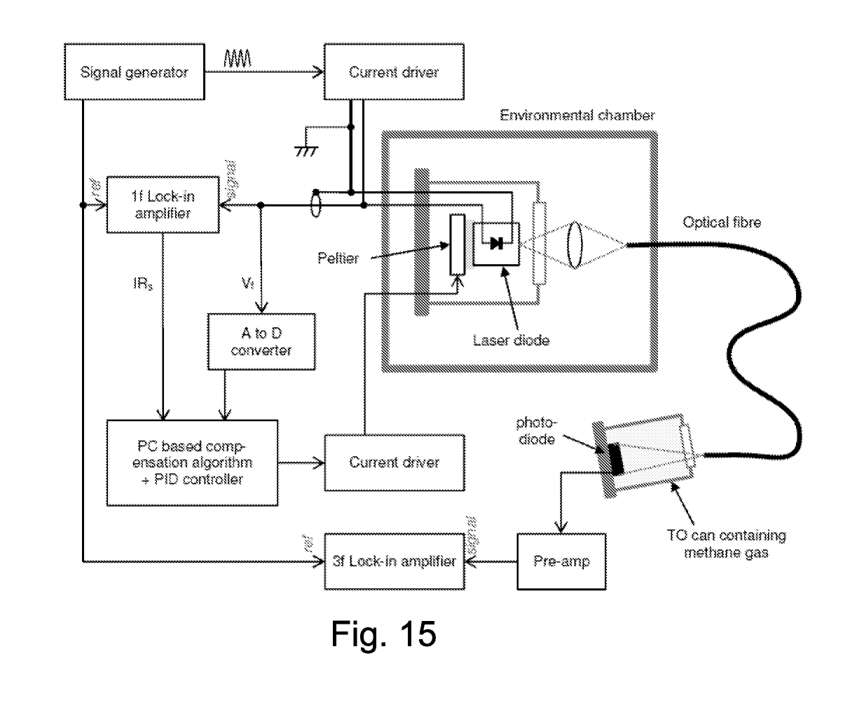

FIG. 15 shows the experimental configuration for laser diode wavelength control using the laser diode junction voltage. The laser diode was modulated at 31 kHz with a peak-to-peak amplitude of 5.2 mA. A current driver (Thorlabs LDC200) was used to bias the diode with DC injection current of 165.8 mA. A lock-in amplifier (Stanford Research SR850) was used to measure the 1f modulated forward voltage, providing a resistance measurement to compensate the forward voltage as shown in FIG. 14. The resulting junction voltage was compared to a setpoint voltage to provide an error signal to the PID. Voltages were measured using a data acquisition card (National Instruments PCI 6259); voltage compensation and the PID were all implemented in Labview. The output from the PID was sent to the modulation input of a second current driver (ThorLabs ITC510) in order to close the feedback loop to the Peltier cooler.

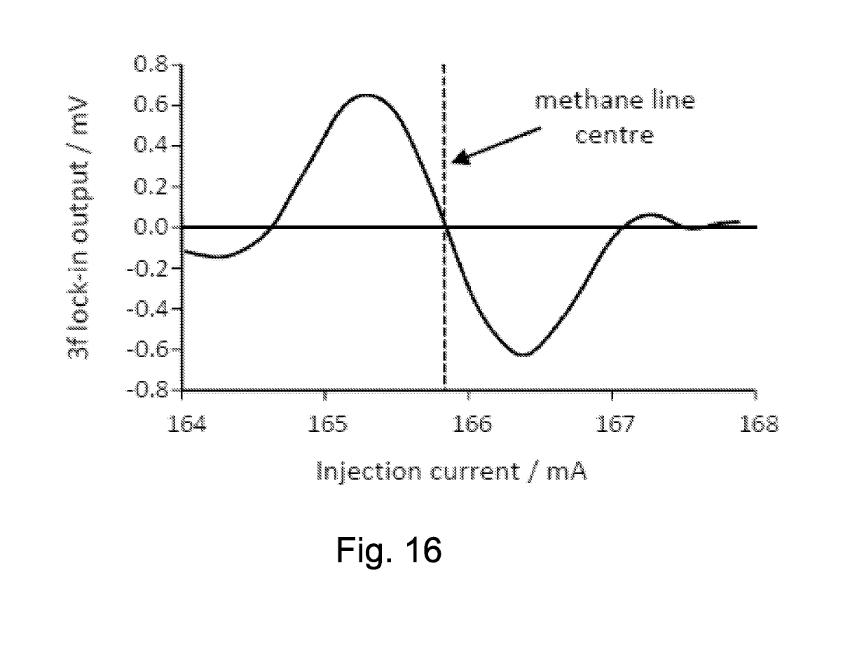

The resolution of an optical spectrum analyser was too coarse to measure the level of wavelength stability resulting from this method, and additionally it would measure the time-averaged emission corresponding to envelope of the wavelength modulation resulting from the applied AC current. Therefore, the laser diode emitted wavelength was measured with the help of a methane reference cell, an 8 mm long TO can filled with methane at approximately 2.5% volume and containing an InGaAs photodiode. The signal from the photodiode pre-amplifier was demodulated at 3f using a lock-in amplifier (Stanford SRS 850) in the manner of wavelength modulation spectroscopy. The 3f signal passes through zero at the gas line centre (see FIG. 16) therefore this signal offered a convenient measure of the deviation of the emitted wavelength from the line centre at 1650.96 nm. In this technique, the deviation of the centre wavelength is measured; the small wavelength modulation means that the wavelength as constantly varying.

Considering now gas reference cell wavelength calibration, a configuration involved a laser diode driven using a sinusoidal signal with frequency 31 kHz and p-p amplitude 5.2 mA, giving a p-p wavelength modulation of 115 pm. A simultaneous DC bias was applied in the range 164-168 mA.

To establish a wavelength calibration, the centre wavelength of the laser diode was scanned across methane gas line while simultaneously applying the sinusoidal modulation. The detector output was then fed into the lock-in amplifier and the third harmonic was measured. The results are shown in FIG. 16. The zero crossing at an applied current of 165.8 mA of the 3f signal corresponds to the peak of the methane gas line. To make sure that the line centre has been identified, the calibration process was repeated while observing the 1f, 2f and 3f signals.

A wavelength calibration was established as follows, giving the rate of change of 3f signal with centre wavelength A.

$$\frac{d ( 3 f signal )}{d ( λ )} = \frac{d ( 3 f signal )}{d ( {I}_{f} )} \times \frac{d ( {I}_{f} )}{d ( λ )} \qquad ( 11 )$$

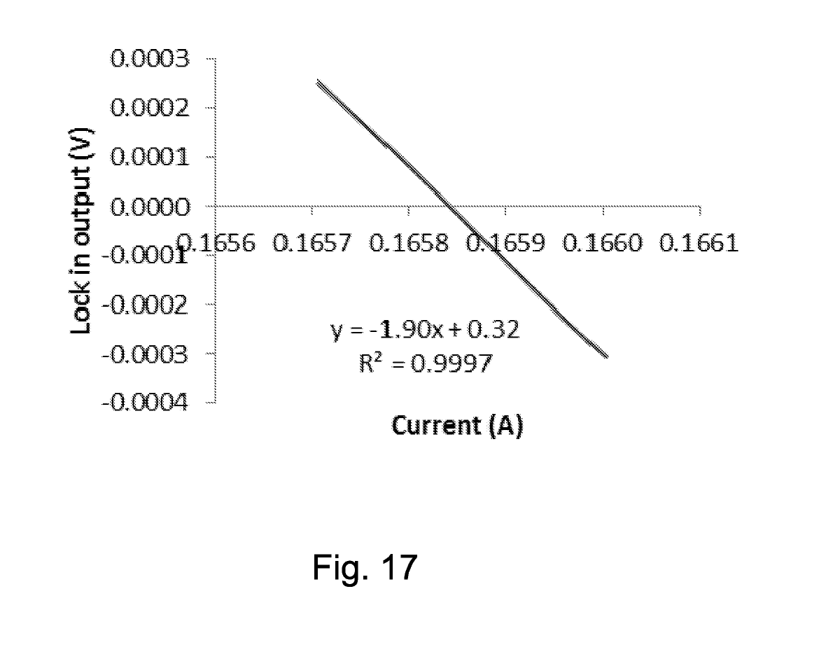

The laser diode wavelength tuning co-efficient, d(λ)/d(I_{f}), was established to be 23 pm/mA by measuring the wavelength at different DC injection currents using the optical spectrum analyser. The value of d(3f signal)/d(I_{f}) was established by expanding the plot in FIG. 16, giving a linear region shown in FIG. 17 below. This gave a gradient of 1.90 V/A.

By this method, the voltage output from the 3f lock-in amplifier was established to have a calibration coefficient of d(3f signal)/d(Δ)=81 μV/pm.

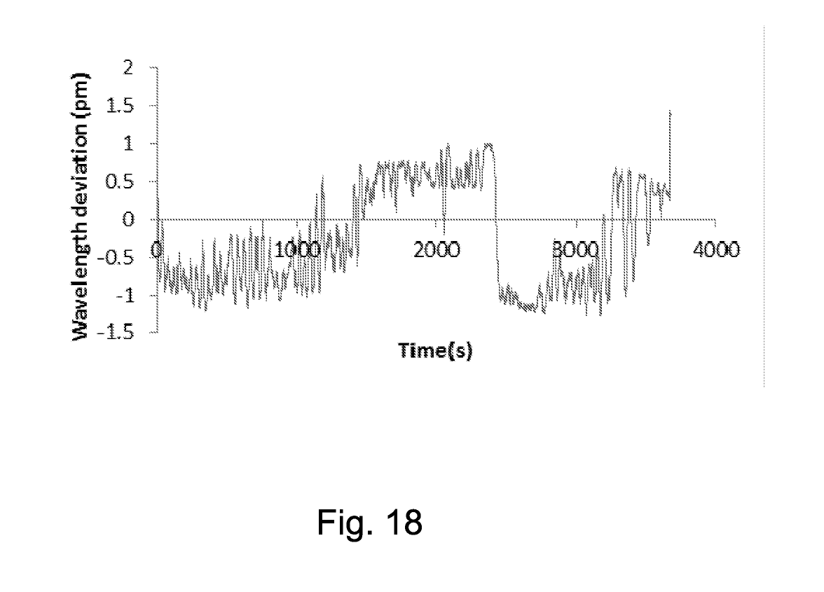

Regarding the results, the laser was operated under junction voltage control for a period of one hour at a constant ambient temperature of 20° C. and a DC injection current of 165.8 mA. The lock-in amplifier 3f signal was recorded and converted into wavelength using the calibration procedure described above in relation to gas reference cell wavelength calibration. FIG. 18 shows the resulting wavelength deviation from the gas line centre over time.

The laser diode remained stable over a period of one hour with a wavelength standard deviation of σ=±0.7 pm. The long-term wavelength stability of the laser diode with forward voltage was therefore comparable to that of the thermistor and forward voltage control methods.

The junction voltage control technique was also tested for wavelength stability with changes in ambient temperature in the range of 15-35° C.

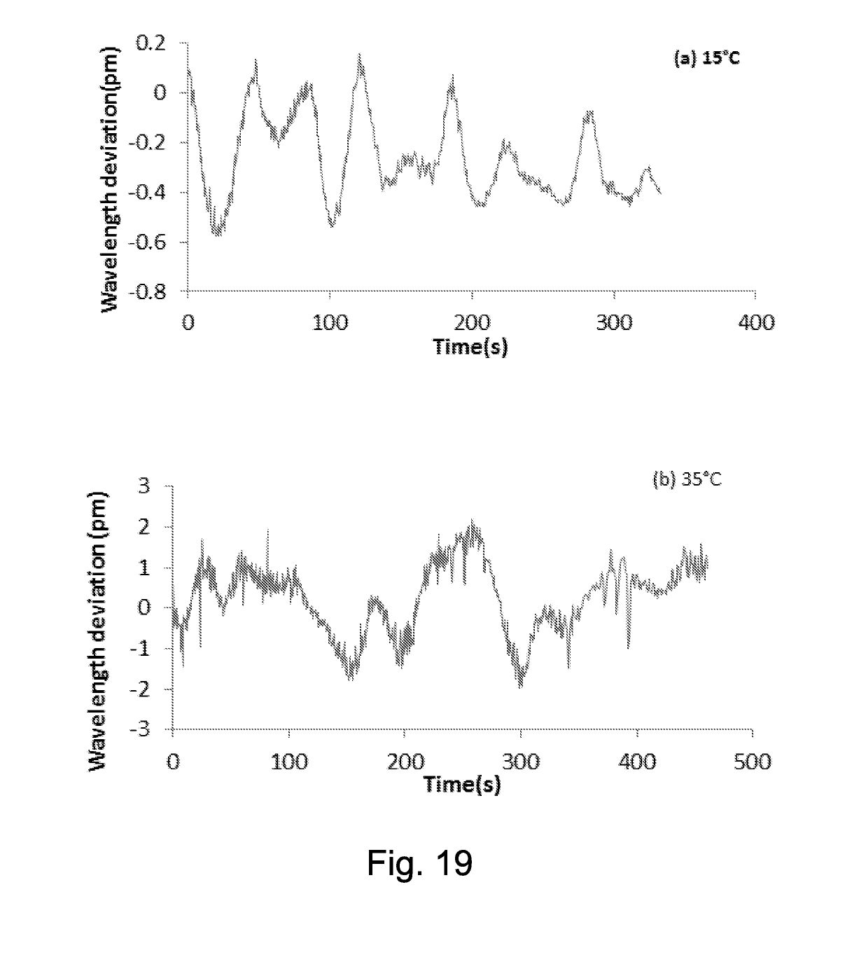

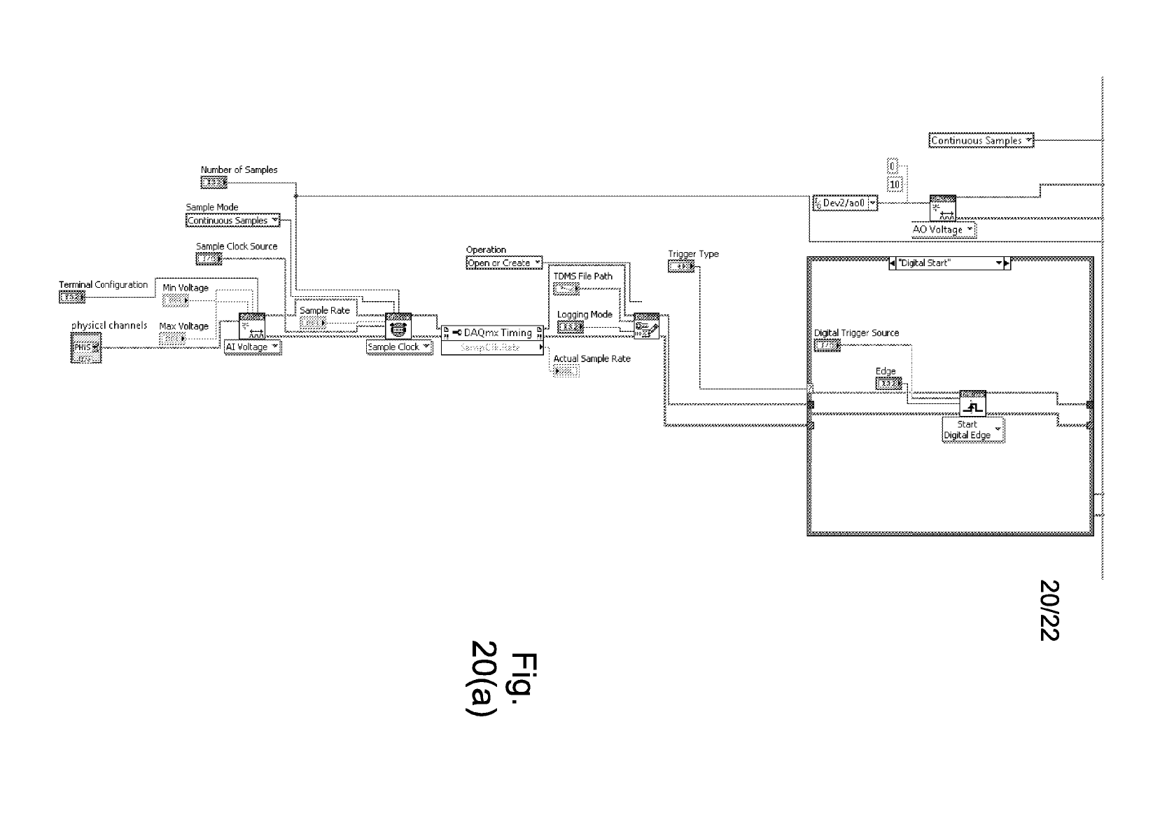

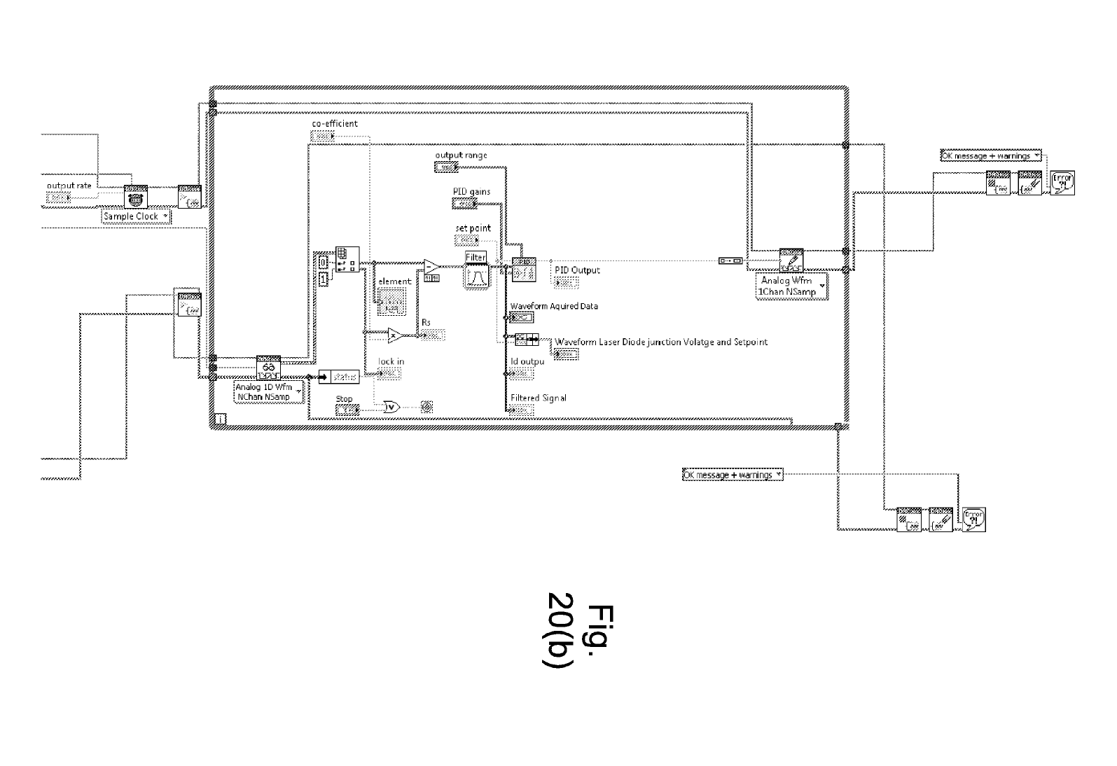

FIG. 19 shows the response of the laser diode wavelength over time and to a change in ambient temperature. In both figures, the 3f voltage output remained close to the zero crossing point, indicating the laser diode wavelength was near the centre of the methane gas line.

Measurements have shown the laser diode wavelength drift with varying ambient temperature. The laser diode wavelength remained stable with ambient temperature coefficient of ≦0.03 pm/° C. The laser diode wavelength stability with ambient temperature using junction voltage is therefore 2 orders of magnitude better than that achieved using conventional thermistor control and using the forward voltage control technique.

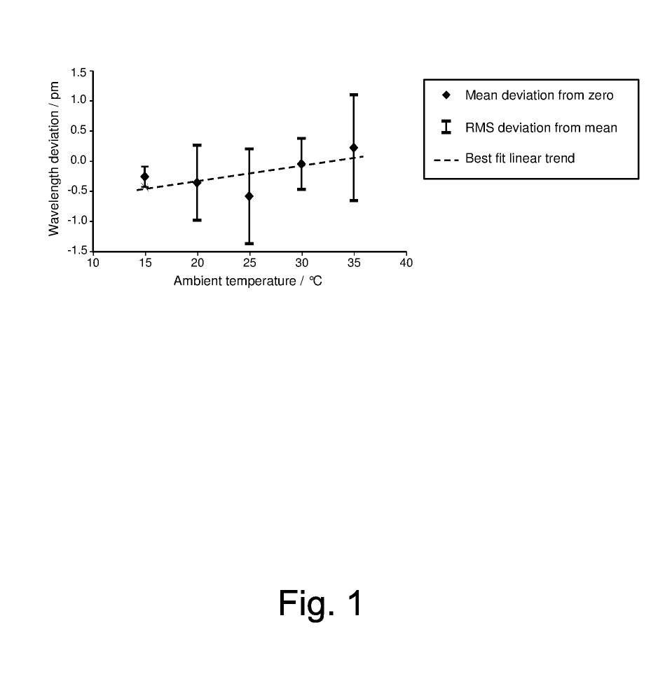

Regarding laser diode centre wavelength stability with ambient temperature under junction voltage control, we refer to FIG. 1.

In view of the above, an approximately linear relationship between DFB laser diode voltage and temperature can be used as a temperature sensor and, within a control loop, to stabilise the central wavelength of the laser diode. The laser diode forward voltage decreased approximately linearly with increasing operating temperature. In conventional laser diode temperature control, a thermistor placed at a distance is used to sense the temperature of the laser diode chip. The thermistor measures the temperature of its immediate surroundings. However, there is a temperature gradient between the chip and the thermistor, resulting in laser diode wavelength drift when the ambient temperature changes. It was experimentally demonstrated that, under conventional thermistor control, the laser diode wavelength decreased with increasing ambient temperature.

The voltage across the laser diode (forward voltage) can be used to stabilise the emission wavelength of the laser diode. The performance of the forward voltage control technique was comparable to that of conventional thermistor control. However, with variable ambient temperatures the laser diode wavelength drifted due to the temperature dependence of the laser diode series resistance. The laser diode forward voltage decreased with increasing temperature, whereas laser diode series resistance increased with increasing temperature. Therefore, when the ambient temperature was increased, the forward voltage decreased, resulting in laser diode wavelength drift towards longer wavelengths.

The laser diode series resistance was measured over a range of operating temperatures. The laser diode resistance increased with increasing temperature, in contrast to a decrease in the forward voltage. A control system was developed based on measurement of the junction voltage of the laser diode to stabilise its wavelength. The laser diode resistance was measured dynamically, by modulating the injection current with a DC bias above threshold. The laser diode was modulated at a high frequency (31 kHz) with a small amplitude to minimise the additional heat generated due to modulation. The 1f demodulated output was scaled and subtracted from the voltage drop measured across the laser diode. The resulting junction voltage was subtracted from a voltage setpoint and the result used as an error signal in a PID control loop.

Due to the limited resolution of the spectrum analyser, a methane gas line was used to check the wavelength stability of the laser diode when using junction voltage control. The centre wavelength of the laser diode was checked for long-term stability and under variable ambient temperatures. The laser diode central wavelength had a long-term stability similar to that of the forward voltage and thermistor control methods, as shown below in Table 2. However, when faced with different ambient temperatures, junction voltage control showed a mean wavelength stability 2 orders of magnitude better than that of either thermistor or forward voltage control.

TABLE 2
Comparison of laser diode wavelength
(or frequency) control techniques

| Control | Long term stability | Wavelength drift with |
|---|---|---|
| technique | (30 min) | ambient temperature |
| Thermistor | ±0.4 pm (±40 MHz) | 3.8 pm/° C. (420 MHz/° C.) |
| control | | |
| Junction | ±0.6 pm (±70 MHz) | ≦0.03 pm/° C. (≦3.3 MHz/° C.) |
| voltage | | |
| control | | |

Tuneable laser diode spectroscopy requires a very stable laser diode wavelength as the gas absorption lines are very narrow, e.g. the methane absorption at 1650.96 nm has a linewidth (HWHM) of 50 pm for 100% concentration. For an ambient temperature change of 20° C., conventional thermistor control would suffer a wavelength change of 76 pm, greater than the methane gas linewidth at 1650.96 nm. Using junction voltage control would bring this down to a more manageable 0.6 pm, which is much smaller than the gas linewidth and similar to the level of wavelength stability over time at a fixed temperature.

The technique is applicable to various packages, e.g., the TO can—packaged DFB described here, a butterfly-packaged DFB and a VCSEL, all at the same or other wavelength, and a UV, visible or IR LED. Generally, the laser may be a distributed feedback laser wherein the active region of the laser comprises a diffraction grating. The laser may be comprise a distributed Bragg reflector, e.g., may be vertical cavity surface emitting laser. The laser may by InP- or GaN-based. The laser may be a quantum dot laser.

No doubt many other effective alternatives will occur to the skilled person. It will be understood that the invention is not limited to the described embodiments and encompasses modifications apparent to those skilled in the art lying within the spirit and scope of the claims appended hereto.

1. Method of controlling emission wavelength of a light emitting device, the method comprising:
determining an indication of series resistance of the device, the series resistance comprising ohmic resistance of the device;
providing a current through the device to maintain light emission of the device;
measuring a forward voltage of the device during said light emission, the forward voltage being across an impedance comprising impedance of an active region of the device and the series resistance;
determining an indicator of active region voltage of the device on the basis of the measured forward voltage and the determined indicator of series resistance; and
controlling a temperature of the device on the basis of the determined indicator of active region voltage.

2. Method of claim 1, wherein the determining the indication of series resistance comprises:
providing an amplitude modulated said current through the device to maintain light emission;
extracting an AC component of forward voltage of the device while providing the amplitude modulated current;
determining a dynamic resistance indication on the basis of the extracted AC component and the modulation amplitude of the amplitude modulated current, to thereby obtain the indication of series resistance.

3. Method of claim 2, comprising inputting the forward voltage to a phase-sensitive detector to perform said extracting the AC component.

4. Method of claim 1, wherein the indication of series resistance is a voltage and:
the measuring the forward voltage of the device comprises detecting a DC forward voltage of the device;
the indicator of active region voltage is determined on the basis of the difference between the detected DC forward voltage and the voltage indication of series resistance.

5. Method of claim 1, comprising:
comparing the measured forward voltage and the indication of the series resistance;
comparing a result of the comparison to a predetermined value, to thereby generate an error signal;
performing said controlling the temperature on the basis of the error signal.

6. Method of claim 1, wherein a maximum wavelength drift of the device, when the ambient temperature of the device varies between 15 and 35 degrees Celsius, is less than +−0.03 pm/degree Celsius, preferably less than +−0.02 pm/degree Celsius.

7. Method of claim 1, wherein a standard deviation of emission wavelength of the device is less than +−0.7 pm, preferably less than +−0.3 pm, at an ambient temperature of 20 degrees Celsius.

8. Method of claim 1, wherein a maximum variation of standard deviation of emission wavelength of the device is less than +−0.6 pm, preferably less than +−0.4 pm, when ambient temperature of the device is varied between 15 and 35 degrees Celsius.

9. Method of claim 1, wherein the light emitting device comprises a light emitting diode or laser, preferably a distributed feedback laser and/or comprising a distributed Bragg reflector or VCSEL.

10. System for wavelength control of a light emitting device, the system comprising:
a current driver to provide an amplitude modulated current through the device to maintain light emission of the device;
a thermoelectric element for controllable heating and cooling of the device;
a DC voltage detector to detect a DC component of forward voltage of the device;
a phase-sensitive detector to extract an AC component of the forward voltage;
a scaling circuit to scale the extracted AC component based on a modulation amplitude of the amplitude modulated current;
a comparator to compare the detected DC component to the scaled extracted AC component, to thereby indicate an active region voltage of the device; and
a temperature controller configured to control the thermoelectric element on the basis of the indication of active region voltage.

11. System of claim 10, wherein the temperature controller comprises a proportional integral and derivative controller configured to control the thermoelectric element on the basis of the indication of active region voltage.

12. System of claim 10, wherein the light emitting device comprises a light emitting diode or laser, preferably a distributed feedback laser and/or comprising a distributed Bragg reflector or VCSEL.

13. Spectrometer comprising the system of claim 10.

14. Spectrometer of claim 13 for detecting one or more gases having absorption spectra that fall within a range of wavelengths emitted by the device.

15. Spectrometer of claim 13 for detecting methane.

16. Method of controlling emission wavelength of a light emitting device, the method comprising:
providing a current through the device to maintain light emission of the device; measuring a forward voltage of the device during said light emission, the forward voltage being across an impedance comprising an active region of the device and series resistance of the device;
determining an indication of temperature of a temperature sensitive device thermally coupled to the light emitting device;
determining a corrected forward voltage on the basis of the measured forward voltage and the determined indicator of temperature; and
controlling a temperature of the light emitting device on the basis of the determined corrected forward voltage.

17. A system for wavelength control of a packaged light emitting device, the system comprising:
a temperature sensitive device thermally coupled to the device package;
a DC voltage detector to detect a DC component of forward voltage of the device;
a comparator to compare a reading from the temperature sensitive device to the detected DC component to give a corrected DC forward voltage; and
a temperature controller configured to control a thermoelectric element on the basis of the indication of corrected DC forward voltage, to thereby control temperature of the light emitting device.
